(19) United States
(12) Patent Application Publication
(10) Pub. No.: US 2026/0077087 A1
(43) Pub. Date: Mar. 19, 2026

(54) COMPOSITION FOR PHOTOCHEMICAL TISSUE BONDING

(71) Applicant: UNIVERSITY OF SOUTH FLORIDA, Tampa, FL (US)

(72) Inventors: Nurettin SAHINER, Wesley Chapel, FL (US); Manoj Kumar RAM, Palm Harbor, FL (US); Ramesh S. AYYALA, Tampa, FL (US)

(21) Appl. No.: 19/108,906

(22) PCT Filed: Sep. 6, 2023
(86) PCT No.: PCT/US2023/032054

§ 371 Date: Mar. 5, 2025

Related U.S. Application Data
(60) Provisional application No. 63/404,005, filed on Sep. 6, 2022.

(51) Int. Cl. A61L 24/08; A61L 24/00; A61L 24/04

(52) U.S. Cl. CPC A61L 24/08; A61L 24/001; A61L 24/0015; A61L 24/046; A61L 2430/34

(57) ABSTRACT

The present disclosure provides for a photocrosslinkable tissue bonding composition comprising: a poly(ethylene glycol) functionalized with at least one photocrosslinkable group, a hydroxy-modified chitosan, and a photochemically active dye. Further provided herein is a photocrosslinked tissue bonding composition comprising: a photochemically active dye and a functionalized poly(ethylene glycol). Also disclosed is a kit for treating a wound, comprising a poly(ethylene glycol) functionalized with at least one photocrosslinkable group, a hydroxy-modified chitosan, a photochemically active dye, and an applicator. Further provided is a method for producing a photocrosslinkable tissue bonding composition and a method for treating a wound.

### CROSS-REFERENCE TO RELATED APPLICATIONS

This application claims the benefit of priority to, and the benefit of, U.S. Provisional Application No. 63/404,005 filed on Sep. 6, 2022, the disclosure of which is hereby expressly incorporated by reference herein in its entirety.

### BACKGROUND

Suturing is a standard procedure for closing wounds. Alternative methods for wound closure have been developed and refined in the past decades, and in recent years there has been increasing interest in improving surgical adhesives and sealants. Surgical adhesives allow patients to experience less pain when undergoing the reconnecting and sealing of tissues, in comparison to the discomfort that is often experienced with scar tissue formation that occurs postoperatively when traditional sutures are used. Surgical adhesives have also been found useful because they cause minimal wound inflammation and are often associated with lower rates of infection in comparison to traditional suture methods.

Another sutureless method for tissue repair that is provided by surgical adhesives is performed through a photochemical tissue bonding (PTB) process. These compositions have some drawbacks, however. Particularly, PTB can lead to tissue decay when too much heat is utilized in the PTB process. For example, a composition comprising albumin did not survive under the PTB process because the composition exceeded a temperature of 70° C., which denatured both the albumin composition and collagen molecules.

Further, polyvinyl alcohol (PVA) has been found to be an alternative material for PTB, but it creates thrombogenic characteristics in the tissues it binds and cannot easily be removed after the repair. Several materials such as naphthalimides, PVA, and albumin have been used alone and in combination as PTB compositions, but each prior composition left residues behind in the bonded tissues and, more detrimentally, lacked biocompatibility and solubility in blood.

Despite the availability of numerous surgical adhesives, they are often met with limited success in effectively treating the wound tissue due to limited biocompatibility, blood solubility, and heat stability. They can also be costly. Accordingly, a need exists for an affordable and biocompatible sutureless tissue bonding adhesive. The methods and compositions disclosed herein address these and other needs.

### SUMMARY

In accordance with the purposes of the disclosed materials and methods, as embodied and broadly described herein, the disclosed subject matter, in one aspect, relates to a composition and methods of making and using thereof.

Thus, in one example, a photocrosslinkable tissue bonding composition is provided, including a poly(ethylene glycol) (PEG) functionalized with at least one photocrosslinkable group, a hydroxy-modified chitosan, and a photochemically active dye.

In a further example, a photocrosslinked tissue bonding composition is provided, including a photochemically active dye and a poly(ethylene glycol), and optionally a hydroxy-modified chitosan, wherein the hydroxy-modified chitosan is entrapped in the photocrosslinked poly(ethylene glycol) and/or grafted to the photocrosslinked poly(ethylene glycol).

Additionally, a kit for treating a wound is provided, including a poly(ethylene glycol) functionalized with at least one photocrosslinkable group, a hydroxy-modified chitosan, a photochemically active dye, and an applicator.

In one example, a method for producing a photocrosslinkable tissue bonding composition is provided, the method comprising preparing a poly(ethylene glycol) functionalized with at least one photocrosslinkable group and a hydroxy-modified chitosan in a fiber or a bulk form.

In a further example, a method for treating a wound is provided, the method comprising contacting the wound with the composition as disclosed herein and irradiating the composition with a light.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The accompanying figures, which are incorporated in and constitute a part of this specification, illustrate several aspects of the disclosure, and together with the description, serve to explain the principles of the disclosure.

FIG. 1 shows an example wound healing process using PTB based hydrogel precursor.

FIG. 2 shows the crosslinking of PEG acrylates for the formation of PEG hydrogel.

FIG. 3 shows the chemical structures of glycol chitosan and poly(ethylene glycol diacrylate), and the oxidation of Rose Bengal and hydrogel.

FIG. 4 shows the chemical structure of a poly(ethylene glycol)-chitosan composite hydrogel.

FIG. 5 shows pictures of poly(ethylene glycol)-chitosan composite hydrogel formation and swelling of particles in the presence of water.

FIG. 6 shows a schematic of an example network of PTB materials.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

In this specification and in the claims that follow, reference will be made to a number of terms, which shall be defined to have the following meanings.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint.

The term “patient” refers to a human in need of treatment for any purpose. However, the term “patient” can also refer to non-human animals, preferably mammals such as dogs, cats, horses, cows, pigs, sheep and non-human primates, among others, which are in need of treatment.

### Compositions

### Photocrosslinkable Tissue Bonding Composition

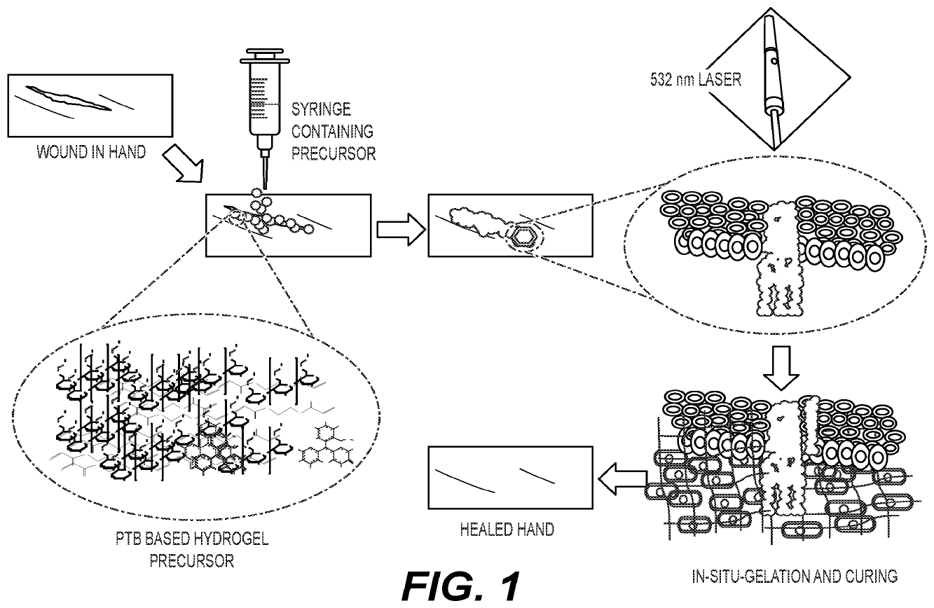

Provided herein is a photocrosslinkable tissue bonding composition comprising a poly(ethylene glycol) functionalized with at least one photocrosslinkable group, a hydroxy-modified chitosan, and a photochemically active dye. The compositions can be dissolved in blood in from ten minutes to one hour, are biocompatible, and can be commercially produced at a reasonable cost. FIG. 1 shows a schematic of healing a wound using the photocrosslinkable tissue bonding compositions disclosed herein.

The photocrosslinkable tissue bonding compositions can be used as surgical adhesives for wound closure or other tissue bonding purposes. The disclosed compositions can reconnect and seal tissues with less pain to patients. The disclosed compositions can also result in minimal wound inflammation and a lower rate of infection than sutures. Methods of using the disclosed compounds for tissue repair can be performed through a PTB process where the disclosed compositions are crossed linked with minimal heat generation through irradiation (e.g., green laser; 532 nm). There should be minimal heat generation under the disclosed processes, thus avoiding decaying the tissues. The resulting composition after photochemically crosslinking the disclosed photocrosslinkable tissue bonding compositions is a poly(ethylene glycol) chitosan composite hydrogel.

Table 1 shows examples of poly(ethylene glycol) s functionalized with at least one photocrosslinkable group, which can be used in the disclosed compositions and methods.

TABLE 1
The structures and properties of select polyethylene glycol-based polymers.

| Chemical | Structure | Properties |
|---|---|---|
| Poly(ethylene glycol) methyl ether acrylate | 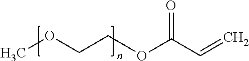 | Liquid, Transparent, B.P 78° C., water soluble |
| Poly(ethylene glycol) dimethacrylate | 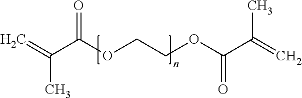 | Powder, color: White- White-off, B.P. >200° C. |
| Poly(ethylene glycol) | 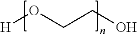 | Solid, M.P 61-64° C. |
| Poly(ethylene glycol) diacrylate | 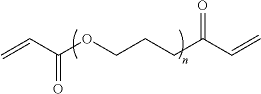 | Liquid, colorless, M.P. 12-17° C., water soluble |
| Poly(ethylene glycol) dithiol | 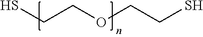 | Solid, M.P 29-33° C. |
| Poly(ethylene glycol) methyl ether thiol | 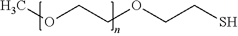 | Solid, Odor: Stench, M.P. 50-55° C. |
| Poly(ethylene glycol) methyl ether amine | 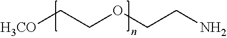 | Color: Light grey, Liquid |
| Poly(ethylene glycol) bis(amine) | 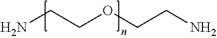 | Solid, M.P. 54-60° C. |
| 2-N-Morpholinoethyl methacrylate | 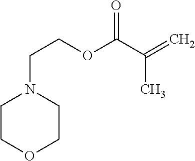 | Liquid, B.P. 78° C. |
| Pentaerythritol tetraacrylate, Pentaerythritol triacrylate, and Trimethylolpropane triacrylate mixture | 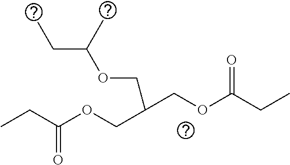 [SMILES: CCC(=O)OCC(COC(=O)CC)COC(C)CC] | Color: yellow, liquid, d: 1,18 g/cm³ |
| | 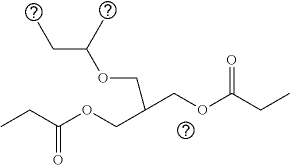 [SMILES: CCC(=O)OCC(COC(=O)CC)COC(C)CC] | |
| | 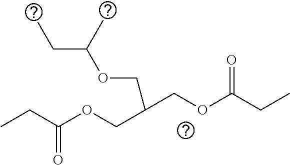 [SMILES: CCC(=O)OCC(COC(=O)CC)COC(C)CC] | |
| Pentaerythritol tetraacrylate | 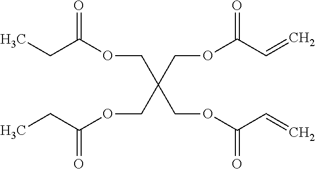 [SMILES: C=CC(=O)OCC(COC(=O)C=C)(COC(=O)CC)COC(=O)CC] | Viscous liquid, colorless d: 1.19 g/cm³ |

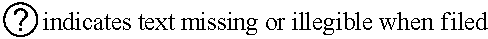

The term “PEG” refers to poly(ethylene glycol). The chemical structure of PEG is H—(O—CH₂—CH₂)n-OH. PEG is also known as polyethylene oxide (PEO) or polyoxyethylene (POE), depending on its molecular weight. PEG usually refers to oligomers and polymers with a molecular mass below 20,000 g/mol. PEGs are prepared by polymerization of ethylene oxide and are commercially available over a wide range of molecular weights from 300 g/mol to 10,000,000 g/mol. Different forms of PEG are also available, depending on the initiator used for the polymerization process. Lower-molecular-weight PEGs are also available as purer oligomers, referred to as monodisperse, uniform, or discrete.

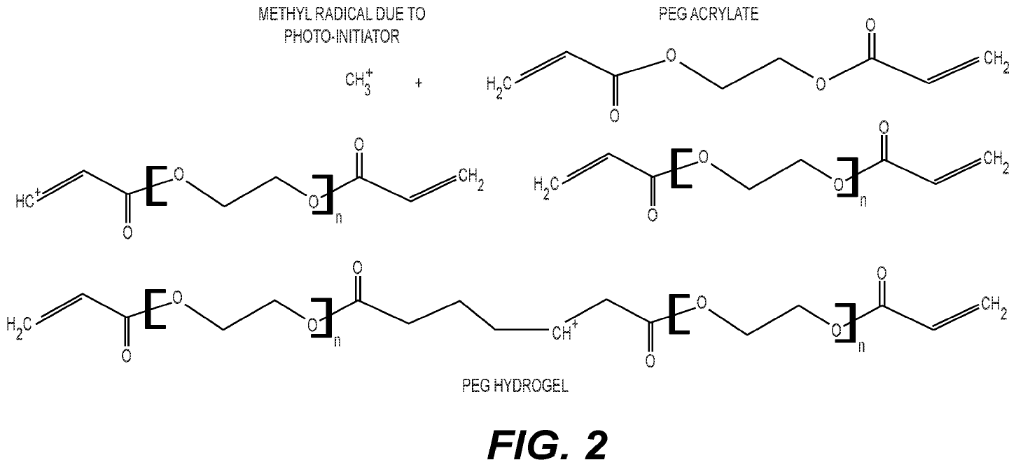

PEG and PEG derivatives for use in the disclosed compositions and methods are water-soluble, biocompatible polymerizable precursors that can contain alkene, epoxide, NH2, SH, OH groups that can be photo crosslinked with UV to visible laser irradiation. In some examples, the derivatives can include PEG containing acrylate as well as amine and/or thiol groups. In further examples, the photo crosslinking of PEG acrylate is in the presence of Rose Bengal photo-initiator to form the based PEG hydrogel. (See FIG. 1, FIG. 2).

The disclosed PEG functionalized with at least one photocrosslinkable group can be combined with a hydroxy functionalized chitosan. Examples of hydroxy functionalized chitosan include a glycol functionalized chitosan. Chitosan is an amino polysaccharide that can be obtained from diverse living organisms. Chitin is obtained from organisms that include, but are not limited to, arthropods, algae, and fungi, and via deacetylation, chitin is converted to chitosan. Chemical production of chitin and chitosan can be applied at stages such as i) deprotonation of the material, ii) demineralization, which is not a necessary step for production from mushroom, iii) discoloration of procured chitin, and finally, iv) chitin deacetylation. Deacetylation of chitin to chitosan can be from either chemical or enzymatic reactions. For example, deacetylation of chitin to chitosan can be carried out with the treatment of 40-60% NaOH at 130-160° C. for 4 hours.

Chitosan is a biopolymer that has broad beneficial characteristic properties including, but not limited to, being biodegradable, biocompatible, and non-toxic. In addition to these features, chitosan as a cationic polymer possesses anti-bacterial, anti-fungal, and anti-viral activity. Low molecular weight of chitosan passes through the bacterial cell wall and prevents mRNA synthesis and DNA transcription and, moreover, the high molecular weight of chitosan can alter the cell wall and blocks the essential substances to go into the bacterial cell. Furthermore, chitosan is a constituent in wound healing and can help skin regeneration on the molecular level. Chitosan also possesses antioxidant activity which can allow for scavenging free radicals and blocking the oxidative sequence. The chitosan derivatives usable herein contain substituents that permit the chitosan derivative to polymerize with the PEG functionalized with at least one photocrosslinkable group to form a composite hydrogen. Glycol chitosan is a chitosan derivative containing hydrophilic ethylene glycol branches and is suitable for use herein.

The term “cross-linked”, “cross-link”, or “cross-linkable” refers to the presence of links or bonds within and/or between chain(s) of the molecules, e.g., chain(s) of chitosan or chitosan derivatives and poly(ethylene) glycol derivatives as a result of a chemical reaction. This can result in a 3D network, interpenetrating network, or graft. “Photocrosslinked” or “photocrosslinkable” refers to cross-linking that is a result of the absorption of energy in the form of light.

“Photochemically active dye”, as used herein, refers to dyes that react upon the absorption of energy in the form of light. Photochemically active dyes can include, but are not limited to, Rose Bengal dye, Riboflavin, Eosin-Y, Erythrosine dye, or any combination thereof.

In some examples, the poly(ethylene glycol) functionalized with at least one photocrosslinkable group comprises poly(ethylene glycol) methyl ether acrylate, poly(ethylene glycol) dimethacrylate, poly(ethylene glycol), poly(ethylene glycol) diglycidyl ether, poly(ethylene glycol) diacrylate, poly(ethylene glycol) dithiol, poly(ethylene glycol) methyl ether thiol, poly(ethylene glycol) methyl ether amine, poly(ethylene glycol) bis(amine), or any combination thereof.

In further examples, the poly(ethylene glycol) functionalized with at least one photocrosslinkable group comprises poly(ethylene glycol) diacrylate and poly(ethylene glycol) methyl ether amine. Poly(ethylene glycol) diacrylate and poly(ethylene glycol) methyl ether amine can have molecular weights of from 0.5 to 40 kDa and 0.5 t0 40 kDa, respectively. In some examples, the molecular weights of the poly(ethylene glycol) diacrylate or poly(ethylene glycol) methyl ether amine can be. 0.5, 1, 5, 10, 15, 20, 25, 30, 35, or 40, where any of the stated values can form an upper or lower endpoint of a range.

In certain examples, the hydroxy-modified chitosan comprises glycol chitosan, hydroxy propyl chitosan, N,O-carboxymethyl chitosan, or any combination thereof.

In specific examples, the photochemically active dye comprises Rose Bengal dye Riboflavin, Eosin-Y, Erythrosine dye, or any combination thereof. The Rose Bengal dye can produce stronger bonding than other dyes, which helps in cross-linking of two tissue segments with a minimal increase in temperature.

Riboflavin (B12) is photoinitiator for the thiol-ene hydrogelation of functionalized poly(ethylene glycol). Double, triple or quadruple bonds containing PEGs with poly(ethylene glycol) dithiol in presence of Riboflavin (B12) can be activated via visible light to prepare hydrogels.

Eosin-Y dye is an organobromine salt that is a 2,4,5,7-tetrabromofluorescein in which the carboxy group and the phenolic hydroxy group have been deprotonated and the resulting charge is neutralized by two sodium ions. It is a fluorochrome and can act as a histological dye. It contains a 2,4,5,7-tetrabromofluorescein (2-).

Erythrosine is a tetraiodofluorescein with the following molecular formula: C₂₀H₈I₄Na₂O₅. It is an organoiodine compound and a derivative of fluorone. Further, it is the disodium salt of 2,4,5,7-tetraiodocluorescein.

Eosin-Y and Erythrosine can also be used as photoinitiators, and in some examples can be used along with some co-initiators as electron donors during the initiation of the polymerization reaction. The electron donor co-initiator can include, but is not limited to, triethylamine, chitosan, chitosan derivatives, or any combination thereof.

In further examples, the composition further comprises 2-N-morpholinoethyl methacrylate, pentaerythritol tetraacrylate, pentaerythritol triacrylate, trimethylolpropane triacrylate, or any combination thereof. The 2-N-Morpholinoethyl methacrylate, pentaerythritol tetraacrylate, pentaerythritol triacrylate, and trimethylolpropane triacrylate are water-soluble precursors that can be cross-linked under Rose Bengal dye, riboflavin, Eosin-Y, or Erythrosine dye with UV to visible laser irradiation.

In certain examples, the poly(ethylene glycol) functionalized with at least one photocrosslinkable group and the hydroxy-modified chitosan are fibers, films, medical textiles, microparticles, or nanoparticles.

In further examples, the poly(ethylene glycol) functionalized with at least one photocrosslinkable group is a water-soluble precursor containing an epoxide, a thiol, an amine group, or any combination thereof.

As used herein, “epoxide” refers to a cyclic ether with a three-atom ring. Examples of epoxides include propylene oxide (PO) and cyclohexene oxide (CHO). “Thiol” refers to a compound comprising an “SH” group, either as the sole functional group or in combination with other functional groups, such as hydroxyl groups. “Amine” or “amine group” refers to a moiety wherein a nitrogen atom is covalently bonded to at least one carbon or heteroatom.

In some examples, the disclosed photocrosslinkable composition is cross-linkable with from 500 to 565 nm of light. In further examples, the composition is cross-linkable at from 500 to 505, 505 to 510, 510 to 515, 515 to 520, 520 to 525, 525 to 530, 530 to 535, 535 to 540, 540 to 545, 545 to 550, 550 to 555, 555 to 560, or 560 to 565 nm. In certain examples, the composition is cross-linkable at from 500 to 520, 520 to 540, or 540 to 565 nm. In specific examples, the composition is cross-linkable at from 500 to 510, 500 to 520, 500 to 530, 500 to 540, 500 to 550, or 500 to 565 nm. In some examples, the composition is cross-linkable at from 500 to 505, 500 to 510, 500 to 515, 500 to 520, 500 to 525, 500 to 530, 500 to 535, 500 to 540, 500 to 545, 500 to 550, 500 to 555, 500 to 560, or 500 to 565 nm.

In further examples, the disclosed compositions can additionally comprise an antibiotic, an antifungal, an anti-inflammatory medication, or any combination thereof.

As used herein, “antibiotic” refers to a substance that is used to treat and/or prevent bacterial infection by killing bacteria, inhibiting the growth of bacteria, or reducing the viability of bacteria. “Antifungal” refers to a substance that kills, destroys, inhibits, or inactivates a fungus. As used herein, “anti-inflammatory medication” refers to a substance that can be used to prevent or reduce an inflammatory response or inflammation in a cell, tissue, organ, or subject.

In certain examples, the antibiotic comprises vancomycin, ciprofloxacin, tetracycline, or any combination thereof.

In specific examples, the antifungal comprises amphotericin B, fluconazole, voriconazole, or any combination thereof.

In some examples, the anti-inflammatory medication comprises acetaminophen, ibuprofen, naproxen, diclofenac, or any combination thereof.

In further examples, the composition has a ratio of the poly(ethylene glycol) functionalized with at least one photocrosslinkable group: hydroxy-modified chitosan of from 1:20 to 1:1, e.g., from 1:15, 1:10, 1:5, 1:2, or 1:1.

In certain examples, the poly(ethylene glycol) functionalized with at least one photocrosslinkable group has a poly(ethylene glycol) methyl ether amine:poly(ethylene glycol) diacrylate ratio of from 0:100 to 100:0, e.g., 0.5:40, 1:10, 10:1, or 100:0.

In specific examples, the poly(ethylene glycol) functionalized with at least one photocrosslinkable group has a molecular weight of from 1 kDa to 10 kDa. In some examples, the molecular weight is from 1 kDa to 2 kDa, 2 kDa to 4 kDa, 4 kDa to 6 kDa, 6 kDa to 8 kDa, or 8 kDa to 10 kDa. In further examples, the molecular weight is from 1 kDa to 2 kDa, 1 kDa to 4 kDa, 1 kDa to 6 kDa, 1 kDa to 8 kDa, or 1 kDa to 10 kDa. In certain examples, the molecular weight is from 1 kDa to 5 kDa or 5 kDa to 10 kDa. In specific examples, the molecular weight is from 1 kDa to 4 kDa, 4 kDa to 7 kDa, or 7 kDa to 10 kDa.

In some examples, the hydroxy-modified chitosan has a molecular weight ranging from 540 g/mol to 810 g/mol. In further examples, the molecular weight ranges from 540 to 560, 560 to 580, 580 to 600, 600 to 620, 620 to 640, 640 to 660, 660 to 680, 680 to 700, 700 to 720, 720 to 740, 740 to 760, 760 to 780, 780 to 800, or 800 to 810 g/mol. In certain examples, the molecular weight ranges from 540 to 570, 570 to 600, 600 to 630, 630 to 660, 660 to 690, 690 to 720, 720 to 750, 750 to 780, or 780 to 810 g/mol. In specific examples, the molecular weight ranges from 540 to 600, 540 to 650, 540 to 700, 540 to 750, or 540 to 810 g/mol. In some examples the molecular weight ranges from 540 to 600, 600 to 700, or 700 to 810 g/mol.

### Photocrosslinked Tissue Bonding Composition

Further provided herein is a photocrosslinked tissue bonding composition comprising: a photochemically active dye and a poly(ethylene glycol)), wherein the poly(ethylene glycol) comprises groups that are photocrosslinked, and a hydroxy-modified chitosan entrapped in the photocrosslinked poly(ethylene glycol) and/or grafted to the photocrosslinked poly(ethylene glycol). In some examples, the photocrosslinked tissue bonding composition can be referred to as a hydrogel.

As used herein, “hydrogel” refers to a cross-linked hydrophilic polymer that does not dissolve in water. Hydrogel can be highly absorbent while maintaining a well-defined structure. Hydrogels can be synthetic or derived from nature. Natural hydrogels include collagen and gelatin. Hydrogels comprise 3D cross-linked polymer networks, which can absorb and retain a large amount of water.

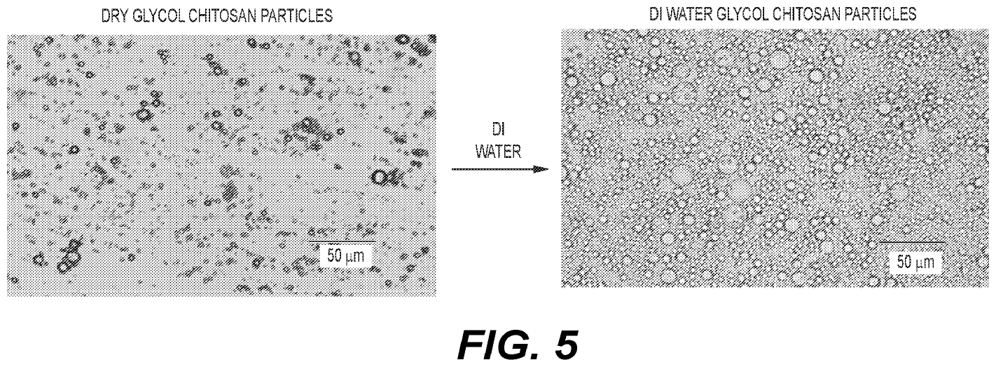

FIG. 5 shows an example mechanism of photocrosslinking functionalized poly(ethylene glycol) with hydroxy-modified chitosan.

In some examples, the poly(ethylene glycol) functionalized with at least one photocrosslinkable group can comprise poly(ethylene glycol) methyl ether acrylate, poly(ethylene glycol) dimethacrylate, poly(ethylene glycol), poly(ethylene glycol) diacrylate, poly(ethylene glycol) dithiol, poly(ethylene glycol) methyl ether thiol, poly(ethylene glycol) methyl ether amine, poly(ethylene glycol) diglycidyl ether, poly(ethylene glycol) bis(amine), or any combination thereof. In some examples, the poly(ethylene glycol) functionalized with at least one photocrosslinkable group can comprise further poly(ethylene glycol) derivatives. In further examples, the photochemically active dye comprises Rose Bengal dye, Riboflavin, Eosin-Y, Erythrosine dye, or any combination thereof.

In certain examples, the hydroxy-modified chitosan comprises glycol chitosan, hydroxy propyl chitosan, N,O-carboxymethyl chitosan, or any combination thereof.

In some examples, the composition is an interpenetrating polymer network or a graft, either of which can be referred to as a composite.

An “interpenetrating polymer network” (IPN) is a polymer comprising two or more networks which are at least partially interlaced on a polymer scale. In some examples, the polymers in an IPN are not covalently bonded to each other. In further examples, the polymers of the IPN are interlaced such that the network cannot be separated unless chemical bonds are broken. IPNs can include semi-interpenetrating polymer networks and pseudo-interpenetrating polymer networks.

As used herein, “graft polymer” refers to segmented copolymers with a linear backbone comprising one composite, and randomly distributed branches comprising another composite.

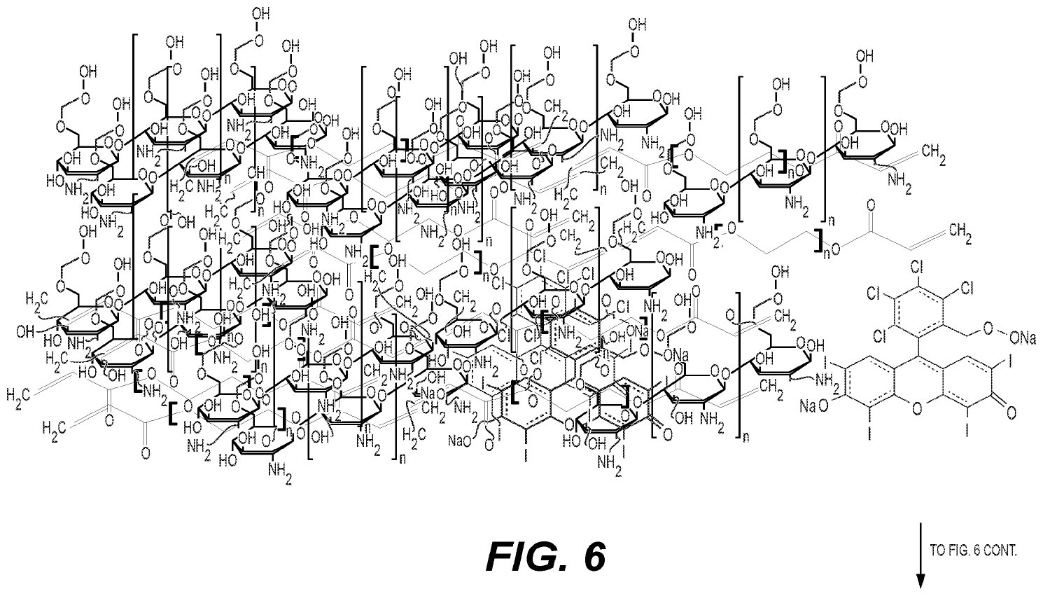

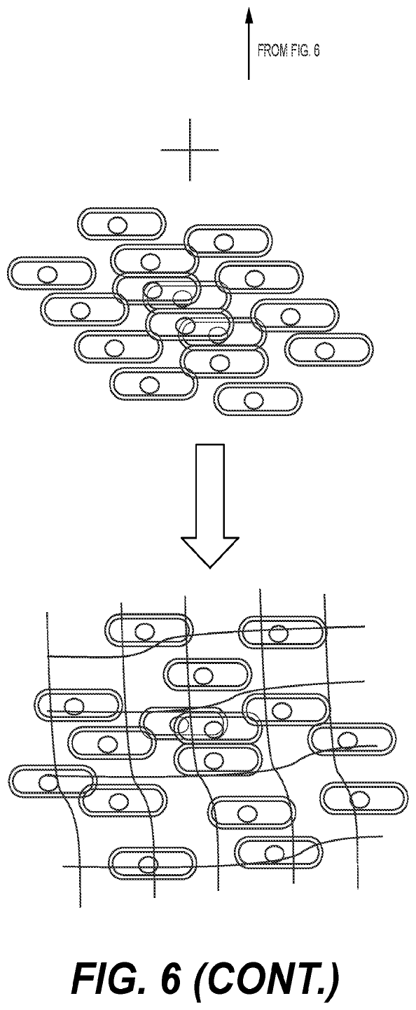

FIG. 5 shows an example network type configuration for the formation of the photocrosslinked tissue bonding composition. FIG. 6 shows an example chemical structure of the photocrosslinked tissue bonding composition.

In further examples, the hydroxy-modified chitosan is cross-linked with pentaerythritol tetraacrylate, pentaerythritol triacrylate, trimethylolpropane triacrylate, or any combination thereof. Pentaerythritol tetraacrylate, pentaerythritol triacrylate, and trimethylolpropane triacrylate are cross-linking agents, which can be used when cross-linking polymers.

In further examples, the composition is formed by reacting the functionalized poly(ethylene glycol) and hydroxy modified chitosan in the presence of the photochemically active dye via visible light. Visible light refers to the segment of the electromagnetic spectrum that the human eye can view. A typical human eye can respond to wavelengths from 380 to 750 nm.

In further examples, the composition can be crosslinked via gamma rays, electron beam, plasma and photochemical, as well as other chemical grafting methods using chain transfer agents in the presence of free radical initiator such as ammonium persulphate and potassium persulfate, as the chain transfer agents can transfer the active radicals into a polymer chain forming a graft structure.

In some examples, the composition is soluble in blood. In further examples, the composition dissolves in blood in from 10 to 60 minutes.

In certain examples, the composition is biocompatible. As used herein, “biocompatible” refers to a composition that performs a desired function with respect to a medical therapy without eliciting any undesirable local or systemic effects.

In some examples, the composition has a swelling percent of from 100% to 1000%, e.g., from 100%, 250%, 500%, 750%, or 1000%. As used herein, “swelling percent” refers to the percent increase in the weight of a hydrogel due to water absorption at a specified time.

In further examples, the composition has a moisture water capacity of from 100% to 1000%, e.g., from 100%, 250%, 500%, 750%, or 1000%. As used herein, “moisture capacity” refers to the percent of the water that is held in the hydrogel at equilibrium.

In certain examples, the composition has a porosity capacity of from 5% to 90%, e.g., from 10%, 20%, 30%, 40%, 50%, 60%, 70%, 80%, or 90%. “Porosity capacity” refers to the percentage of void space in the hydrogel, and is calculated by dividing the volume of the voids by the total volume of the hydrogel.

In specific examples, the composition has an antioxidant activity comparable to some phenolic or natural flavonoid antioxidant compound routine or quercetin. As used herein, “antioxidant activity” refers to a limitation or inhibition of nutrient oxidation, particularly lipids and proteins, by restraining oxidative chain reactions.

The compositions disclosed herein can be formulated into fibers, films, mat and medical textiles, or micro and nano particles.

### Kit for Treating a Wound

Further provided herein is a kit for treating a wound, comprising a poly(ethylene glycol) functionalized with at least one photocrosslinkable group, a hydroxy-modified chitosan, a photochemically active dye, and an applicator.

As used herein, “an applicator” refers to any device that can be used to deliver the elements of the kit to a wound, specifically for treating the wound.

In some examples, the poly(ethylene glycol) functionalized with at least one photocrosslinkable group comprises poly(ethylene glycol) methyl ether acrylate, poly(ethylene glycol) dimethacrylate, poly(ethylene glycol), poly(ethylene glycol) diglycidyl ether, poly(ethylene glycol) diacrylate, poly(ethylene glycol) dithiol, poly(ethylene glycol) methyl ether thiol, poly(ethylene glycol) methyl ether amine, poly(ethylene glycol) bis(amine), or any combination thereof.

In further examples, the poly(ethylene glycol) functionalized with at least one photocrosslinkable group comprises poly(ethylene glycol) diacrylate, poly(ethylene glycol) methyl ether amine, or any combination thereof.

In certain examples, the hydroxy-modified chitosan comprises glycol chitosan, hydroxy propyl chitosan, N,O-carboxymethyl chitosan, or any combination thereof. In specific examples, the photochemically active dye comprises a Rose Bengal dye, Riboflavin, Eosin-Y, Erythrosine dye, or any combination thereof.

### Method

### Method for Producing a Photocrosslinkable Tissue Bonding Composition

Further provided herein is a method for producing a photocrosslinkable tissue bonding composition, comprising preparing a poly(ethylene glycol) functionalized with at least one photocrosslinkable group and a hydroxy-modified chitosan.

In some examples, the hydroxy-modified chitosan is in a fiber or a bulk form.

In further examples, the method further comprises combining the hydroxy-modified chitosan with an antibiotic, antifungal, anti-inflammatory medication, or any combination thereof.

In certain examples, the antibiotic comprises vancomycin, ciprofloxacin, tetracycline, or any combination thereof.

In specific examples, the antifungal comprises amphotericin B, fluconazole, voriconazole, or any combination thereof.

In some examples, the anti-inflammatory medication comprises ibuprofen, naproxen, diclofenac, or any combination thereof.

### Method for Treating a Wound

Further provided herein is a method for treating a wound, comprising contacting the wound with the photocrosslinkable tissue bonding composition as disclosed herein, and irradiating the composition with a light.

As used herein, “irradiate” refers to exposing a composition to light, such as to cause polymerization, which includes, but is not limited to, cross-linking.

In some examples, the hydroxy-modified chitosan comprises glycol chitosan, the poly(ethylene glycol) functionalized with crosslinkable groups comprises poly(ethylene glycol), and the photochemically active dye comprises Rose Bengal dye.

In some examples, the light is green light. Green light refers to light with a peak wavelength of from 500 to 600 nanometers (nm). Green light fills the gap between blue and red light in the visible light spectrum.

In further examples, the irradiation occurs at from 500 to 565 nm. In some examples, the irradiation occurs at from 500 to 505, 505 to 510, 510 to 515, 515 to 520, 520 to 525, 525 to 530, 530 to 535, 535 to 540, 540 to 545, 545 to 550, 550 to 555, 555 to 560, or 560 to 565 nm. In certain examples, the irradiation occurs at from 500 to 520, 520 to 540, or 540 to 565 nm. In specific examples, the irradiation occurs at from 500 to 510, 500 to 520, 500 to 530, 500 to 540, 500 to 550, or 500 to 565 nm. In some examples, the irradiation occurs at from 500 to 505, 500 to 510, 500 to 515, 500 to 520, 500 to 525, 500 to 530, 500 to 535, 500 to 540, 500 to 545, 500 to 550, 500 to 555, 500 to 560, or 500 to 565 nm.

In certain examples, irradiation occurs at 532 nm.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### Examples

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Testing Properties of PTB Composition

Glycol chitosan and various ratios of poly(ethylene glycol) acrylate derivatives were used. Initially, the chemicals were procured commercially. The experiment of mixing the composition with Rose Bengal was initially performed in pH 7.4 buffer and a laser of 532 nm was used for photo-crosslinking. Properties such as swelling pore size, gelation timing, viscosity, mechanical properties, optical properties, degradation, and biocompatibility were tested and optimized.

Selection of Poly(Ethylene Glycol) Derivative at Different Compositions with Glycol Chitosan

Poly(ethylene glycol) diacrylate and poly(ethylene glycol) methyl ether amine-based were selected based on their properties, such as water solubility and highly cross-linking behaviors along with glycol chitosan.

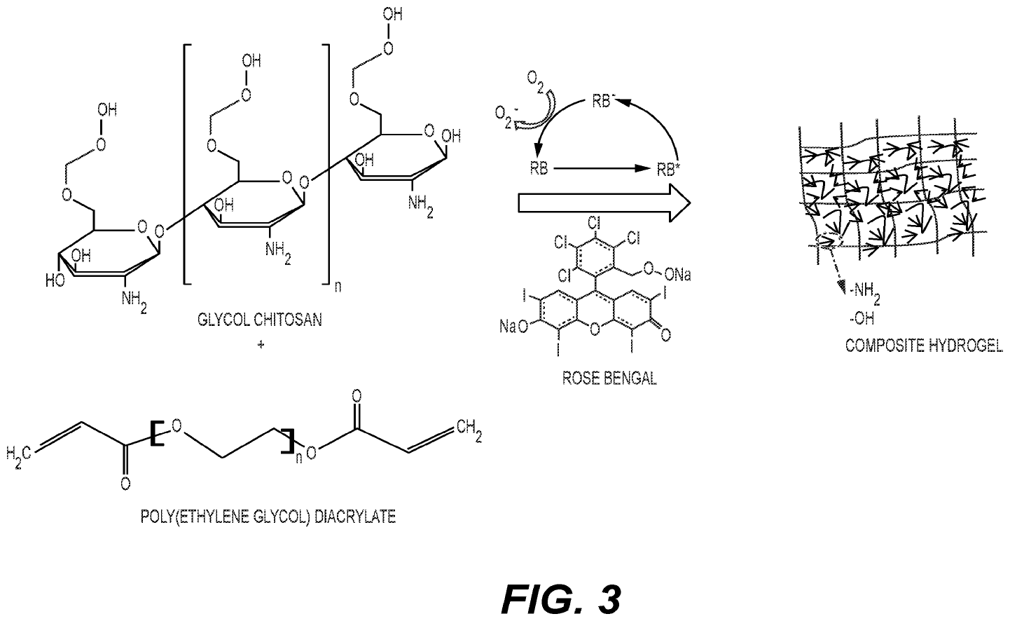

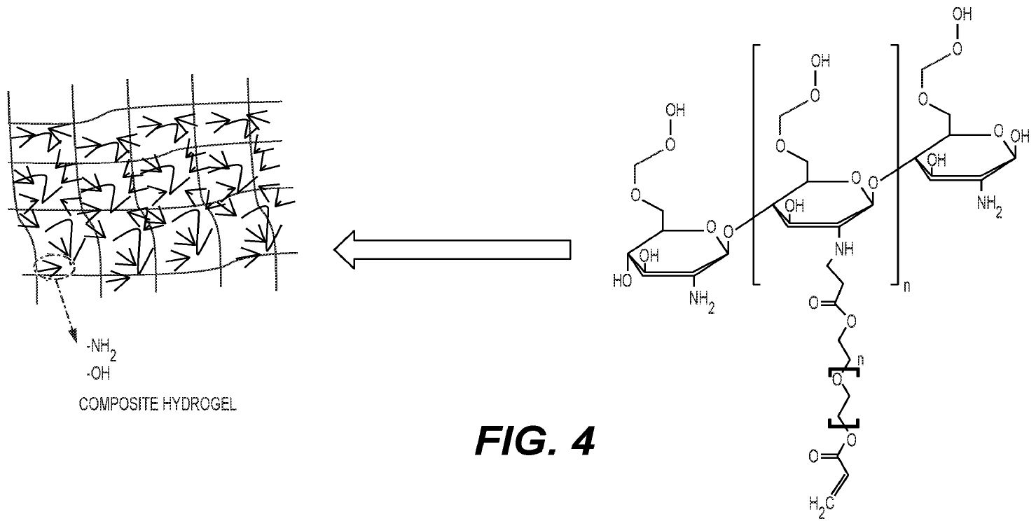

The poly(ethylene glycol) diacrylate, as well as poly(ethylene glycol) methyl ether amine at different ratios with pH 7.4 PBS, were prepared with Rose Bengal. They were cross-linked and polymerized with a laser. (See FIG. 3, FIG. 4.)

Inclusion of Photosensitizer Molecule with PTB Materials for Simultaneous Polymerization, Cross-Linking, and Optimization

PEG derivative with glycol chitosan and Rose Bengal dye were used to understand the photochemical reaction.

The cross-linked materials' optical and mechanical properties were characterized.

Each precursor was also performed separately to understand the crosslinking with optical characterization.

### Hydrogel Formation and Characterization

The glycol chitosan and PEG derivative with Rose Bengal at different ratios were irradiated with a laser of 532 nm. There was crosslinking between the precursors and the hydrogel was formed, which was then characterized to understand the properties of the material with Rose Bengal dye at different ratios.

### Swelling and Degradation Studies

The swelling behavior studies such as the swelling percent (S %), moisture capacity (M %), and porosity percent (P %) of hydrogels were determined at pH 7.4 phosphate buffer solution (PBS). The dried hydrogel pieces were weighted and immersed in PBS at pH 7.4. Then, the hydrogel pieces were removed from the PBS solution at certain time intervals, and surface water was removed with filter paper. The hydrogel pieces were weighed and their swelling behavior against time was constructed. The swelling percent (S %) and moisture capacity (M %) of the prepared hydrogels were calculated using Eq. (1) and Eq. (2). To determine the porosity percent (P %) of the hydrogels, the same hydrogel pieces were squeezed and weighed again. The porosity percent (P %) of the hydrogels was calculated according to Eq. (3).

$$S \% = ( ( {m}_{t} - {m}_{d} ) / {m}_{d} ) \times 100 \qquad ( 1 )$$

$$M \% = ( ( {m}_{s} - {m}_{d} ) / {m}_{s} ) \times 100 \qquad ( 2 )$$

$$P \% = ( ( {m}_{s} - {m}_{sq} ) / {m}_{s} ) \times 100 \qquad ( 3 )$$

wherein, “mₜ” is the weight of swollen hydrogels at time “t”, “mₛ” is the weight of the swollen hydrogel piece at equilibrium, “m_{d}” is the weight of the dry hydrogel piece and “m_{sq}” is the mass of water swollen hydrogel after squeezed.

To determine the pore volume (Vₚ) of the hydrogels, the dried hydrogel piece of known weight was immersed in cyclohexane, was kept immersed for 2-4 hours, and was reweighed. The pore volume of the hydrogels was calculated using Eq. (4).

$$Vp = ( {m}_{ch} - {m}_{d} ) / ( {m}_{d} \times {d}_{ch} ) \qquad ( 4 )$$

wherein, “m_{ch}” is the weight of hydrogel swollen in cyclohexane and “d_{ch}” is the density of cyclohexane.

The gel content was determined by weighing the hydrogels just after preparation and washing the hydrogels exhaustively with distilled water to remove loose polymer chains and unreacted reactants. Then, the washed hydrogels were again dried and reweighed. The ratio between the weight before and after the washing process yielded the gel content and was calculated using Eq. (5).

$$Gel content \% = ( {m}_{a} / {m}_{b} ) \times 100 \qquad ( 5 )$$

wherein “mₐ” is the weight of the dried hydrogel piece after washing and “m_{b}” is the weight of the dried hydrogel piece before washing. All the measurements were completed five times and the results were presented as averages with standard deviations.

### Degradation Studies of Hydrogels

### Hydrolytic Degradation

Hydrolytic degradation of the prepared hydrogels was investigated via the procedure reported in the literature. (Demirci et al., 2020.) Hydrolytic degradation is the degradation of hydrogels in PBS at pH 7.4. This degradation of physiological conditions was in the absence of enzymes. In the hydrolytic degradation studies, hydrogel pieces with known weight were placed in closed tubes with 25 mL of PBS at pH 7.4. These closed tubes were placed in a shaking water bath at 37.5° C. The hydrolytic degradation of the hydrogels was gravimetrically determined after drying the hydrogels taken from the medium at certain time intervals, e.g., 1 hour, 6 hours, 1 day, 3 days, 1 week, etc. and were dried in an oven at 50° C. Hydrolytic degradation % is provided as a function of time using Eq. (6).

$$Hydrolytic degradation \% = ( ( {m}_{0} - {m}_{t} ) / {m}_{0} ) \times 100 \qquad ( 6 )$$

wherein, “m₀” is the weight of the dry hydrogel, and “mₜ” is the weight of hydrogels taken from PBS at different various intervals. The hydrolytic degradation studies were repeated three times and the results were presented as average values with standard deviations.

### Enzymatic Degradation of Hydrogels

For the enzymatic degradation of hydrogels, three types of enzymes such as lysozyme, papain, and lipase were used according to the reported studies in literature. (Shiner et al., 2016; Ari et al., 2020.)

### In the Presence of Enzyme

The enzymatic degradation of the hydrogels in the presence of the Lysozyme enzyme were carried out via the following literature. (Spector et al., 2009.) The prepared, washed, dried weight-known hydrogel pieces were placed in 25 mL of 4 μg/mL lysozyme in pH 7.4 PBS (with 1% penicillin-streptomycin) and were incubated at 37° C. under mild shaking. The enzymatic degradation of hydrogels was gravimetrically determined via weighing hydrogels at certain time intervals, 1 h, 6 h, 1 day, 1-week etc. after drying in an oven at 50° C. The enzymatically degraded amounts of hydrogels by the Lysozyme enzyme were presented as enzymatic degradation % as a function of time following Eq. (6). The degradation studies were repeated three times, and the results were presented as average values with standard deviations. Similarly, the enzymatic degradation of hydrogels in the presence of Papain or Lipase enzymes were carried out according to the literature. (Ari et al., 2020.)

### Antibacterial, Antibiofilm and Antioxidant Properties of Hydrogels

Anti-bacterial and anti-fungal activity of the hydrogels were examined against E. coli (gram−, ATCC 8739), P. aeruginosa (gram−, ATCC 10145), and K. pneumoniae (gram−, ATCC 700603) as gram-negative (−) bacteria, S. aureus (gram+, ATCC 6538), B. subtilis (gram+, ATCC 6633) as gram positive (+) bacteria, C. albicans (ATCC 10231) and Mucormycosis spp. (obtained from a patient) as fungi. The microorganisms were revived from stocks at −20° C. to room temperature in nutrient broth media. The microorganism cultures were transferred into the nutrient broth and incubated at 35° C. overnight. Then, the microorganisms were adjusted according to McFarland 0.5 standard to 1×10⁸ colony-forming unit (CFU)/mL. The anti-microbial studies were investigated by employing both broth macro-dilution and disc diffusion methods.

### Broth Macro-Dilution Method

Different amounts of hydrogels were weighed and sterilized under UV light at 420 nm for 3 minutes. After sterilization, the hydrogels were transferred into 10 mL of nutrient broth and 0.1 mL of adjusted microorganisms were added in hydrogel contained nutrient broths. These nutrient broths were incubated in 35° C. ovens for 18-24 hours. The next day, 0.1 mL of both hydrogel and microorganism contained nutrient broths were seeded on nutrient agar for bacteria and potato-dextrose agar for fungi to be able to count microorganism colonies. The nutrient agar and potato-dextrose agars were incubated in 35° C. ovens for 24 hours. The following day, the bacteria and fungi colonies were counted.

### Antioxidant Studies

The antioxidant properties were investigated from the samples that were taken from the degradation solutions of the hydrogel pieces at different time intervals. The determination of antioxidant behaviors of hydrogels was done by using five different methods: ABTS⁺ scavenging, total phenol content (TPC), total flavonoid content (TFC), Fe(II) chelating capability, and finally FRAP assay. (Ong et al., 2008; Chen et al., 2021; Sudarshan et al., 1992.)

### ABTS⁺ Scavenging Assay

The antioxidant activity of hydrogels was determined by Trolox Equivalent Antioxidant Capacity (TEAC) method, also known as ABTS⁺ radical scavenging assay, with some modifications. (Gohil et al., 2021.) A solution was prepared by mixing 2.5 mL of 2.45 mM potassium persulfate and 7.5 mL of 7 mM ABTS (2,2′-azino-bis-(3-ethylbenzothioazoline-6-sulfonic acid)) solution in DI water. The mixture was left at 4° C. for 12-16 hours in the dark. The stock solution was diluted with PBS until the absorbance decreased to 650-750 and was measured by UV-VIS spectroscopy at 734 nm wavelength. Some degradation solution hydrogels were put in 3 mL of ABTS solution separately and reacted for 6 minutes.

The absorbance of the bare ABTS solution was accepted as blank (A_{blank}), and the ABTS solution (Aₛₐₘₚₗₑ) containing hydrogels after 6 minutes were the samples. The antioxidant scavenging activity of hydrogels was calculated with Eq. (7).

$$Inhibition \% = ( ( {A}_{blank} - {A}_{sample} ) / {A}_{blank} ) 100 \qquad ( 7 )$$

The amount of antioxidant capacity was determined for the values of 20% to 80% reduction of the blank absorbance. Trolox equivalent antioxidant capacity was assessed against the slope of Trolox standard curves and defined as “mM Trolox/g sample”.

### Biocompatibility Test

For the biocompatibility analysis of the hydrogels, MTT cytotoxicity test was performed on L929 fibroblast (Mouse C3/An connective tissue) and HTB-9 (Grade II carcinoma, urinary bladder) cancerous cells. (Dal Pozzo et al., 2000.) In the culture of these cells, Dulbecco's Modified Eagle's Medium (DMEM) supplemented with 10% (v/v) fetal bovine serum (FBS) and 1% penicillin/streptomycin medium and RPMI (w/o: L-Glutamine, w: 2.0 g/L NaHCO₃) supplemented with 10% (v/v) FBS and 1% penicillin/streptomycin medium was used for L929 fibroblast cells and HTB-9 cancerous cells, respectively at 37° C. in a 5% CO₂ atmosphere. In brief, a density of 5×10⁴ cells per well was seeded in a 96-well plate and incubated at 37° C. with 5% CO₂ for 24 h. Then, 5, 10, 15, and 20 mg of the hydrogel was put into the wells containing 100 mL of the fresh culture media to interact with the adherent cells. The plate was incubated at 37° C. with 5% CO₂ for 24 h more. As a control, only 100 mL of culture media was added to the wells. Then, the hydrogel containing culture medium was removed from the wells and washed with PBS. After that, 100 mL of 0.25 mg/mL concentration of MTT agent (3-(4,5-dimethylthiazol-2-yl)-2,5-diphenyltetrazolium bromide) in culture media was placed into the wells and incubated in a dark condition for 2 h. Then, this solution was taken from the wells and 200 mL of dimethyl sulfoxide was added to each well to dissolve formazan crystals. The absorbance of the wells was measured by using a plate reader (Thermo, Multiskan Sky) at 590 nm and the cell viability % was calculated according to the following Eq. 9:

$$Cell viability \% = ( {A}_{s} / {A}_{c} ) \times 100 \qquad ( 9 )$$

wherein, Aₛ and A_{c} are the absorbance of the wells interacted with the hydrogels and non-interacted with the hydrogels as a control, respectively. All analysis was performed in triplicate and the results were given with standard deviations.

### Blood Evaluation Testing

### Analytical Chemical Testing

This testing identified the leaching chemicals from the hydrogel that might interact with a patient. The samples were sent to Nelson Labs to perform the testing and the samples underwent the compound screener test to avoid the interaction of unidentified compounds.

### Compatibility and Tissue Engineering.

For the hemocompatibility tests, the blood was taken from the animals according to the procedures approved by the Clinical Research Ethics Committee and placed into EDTA-containing tubes. These hemolysis and blood clotting assays were determined based on the method proposed by previous processes. (Su et al., 2005.) For hemolysis assay, 2 mL of the blood was diluted with 2.5 mL of 0.9% saline solution, and 200 μL was suspended in 10 mL of 0.9% saline solution. Then, 10 mg of the hydrogels was placed into the blood solution and slowly turned top-down. As a negative and positive control, 200 mL of diluted blood were dispersed in 10 mL of 0.9% saline solution and DI water, respectively. These tubes were incubated in a 37.5° C. water bath for 1 h. After the incubation, the solutions were centrifuged at 100 g for 5 min and the absorbance of the supernatant solution was monitored by using UV-Vis Spectroscopy at 542 nm. The hemolysis ratio % of the hydrogels was evaluated by using Eq. 10:

$$Hemolysis ratio \% = ( ( {A}_{s} - {A}_{n} ) / ( {A}_{p} - {A}_{n} ) ) \times 100 \qquad ( 10 )$$

wherein, Aₛ shows the absorbance of the hydrogel containing blood solution, and Aₙ and Aₚ are the absorbance of the blood solution in 0.9% saline solution and DI water, respectively.

In the blood clotting assay, 64.8 μL of 0.2 M CaCl₂) aqueous solution was mixed with 810 μL of EDTA containing blood and 270 μL of this blood mixture as immediately dropped on 10 mg of the hydrogel, which was placed into flat bottom tubes. These tubes were incubated in a 37.5° C. water bath for 10 min and 10 mL of DI water was gently added into the tubes and centrifuged at 100 g for 1 minute. The supernatant solution was dispersed in 40 mL of DI water and incubated at 37.5° C. for 1 h more. As a control, 250 μL of the blood mixture was dispersed in 50 mL of DI water and incubated at the same conditions. The absorbance of the blood solution was measured by using UV-Vis Spectroscopy at a wavelength of 542 nm. The blood clotting index of the hydrogels was determined by Eq. (11):

$$Blood clotting index \% = ( {A}_{s} / {A}_{c} ) \times 100 \qquad Eq . ( 11 )$$

wherein, Aₛ and A_{c} are the absorbance of the blood solution interacting with the hydrogels and only blood solution without the hydrogels, respectively. All the measurements were performed in triplicate and the results were presented with standard deviations. FIG. 6 shows the schematic of the network of PTB materials under the blood particles.

### Development of the Hydrogel Generation in PBS

The PTB composition developed under this project was optimized and tested. The precursor PEG diacrylate and glycol chitosan with photosensitizer Rose Bengal was developed. This formulation was tested using green light. The materials with blood were treated with laser irradiation to demonstrate the suitability of the sutureless wound closure.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods and compositions of the appended claims are not limited in scope by the specific methods and compositions described herein, which are intended as illustrations of a few aspects of the claims and any methods and compositions that are functionally equivalent are intended to fall within the scope of the claims. Various modifications of the methods and compositions in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

### REFERENCES

A. Lauto, D. Mawad, M. Barton, A. Gupta, S. C. Piller, J. Hook, Photochemical tissue bonding with chitosan adhesive films, Biomedical engineering online, 9 (2010) 1-11.
D. Spector, Y. Rabi, I. Vasserman, A. Hardy, J. Klausner, M. Rabau, A. Katzir, In vitro large diameter bowel anastomosis using a temperature controlled laser tissue soldering system and albumin stent, Lasers in Surgery and Medicine: The Official Journal of the American Society for Laser Medicine and Surgery, 41 (2009) 504-508.
N. M. Ospina, S. P. O. Alvarez, D. M. E. Sierra, D. F. R. Vahos, P. A. Z. Ocampo, C. P. O. Orozco, Isolation of chitosan from Ganoderma lucidum mushroom for biomedical applications, Journal of Materials Science: Materials in Medicine, 26 (2015) 135.
I. Aranaz, M. Mengíbar, R. Harris, I. Paños, B. Miralles, N. Acosta, G. Galed, Á. Heras, Functional characterization of chitin and chitosan, Current chemical biology, 3 (2009) 203-230.
S.-Y. Ong, J. Wu, S. M. Moochhala, M.-H. Tan, J. Lu, Development of a chitosan-based wound dressing with improved hemostatic and antimicrobial properties, Biomaterials, 29 (2008) 4323-4332.
C.-Y. Chen, Y.-C. Chung, Antibacterial effect of water-soluble chitosan on representative dental pathogens Streptococcus mutans and Lactobacilli brevis, Journal of Applied Oral Science, 20 (2012) 620-627.
N. Sudarshan, D. Hoover, D. Knorr, Antibacterial action of chitosan, Food Biotechnology, 6 (1992) 257-272.
M. Z. Elsabee, E. S. Abdou, Chitosan based edible films and coatings: A review. Materials Science and Engineering: C, 33 (2013) 1819-1841.
C. H. Su, S. H. Liu, S. Y. Yu, Y. L. Hsieh, H. O. Ho, C. H. Hu, M. T. Sheu, Development of fungal mycelia as a skin substitute: characterization of keratinocyte proliferation and matrix metalloproteinase expression during improvement in the wound-healing process, Journal of Biomedical Materials Research Part A: An Official Journal of The Society for Biomaterials, The Japanese Society for Biomaterials, and The Australian Society for Biomaterials and the Korean Society for Biomaterials, 72 (2005) 220-227.
P.-J. Park, J.-Y. Je, S.-K. Kim, Free radical scavenging activity of chitooligosaccharides by electron spin resonance spectrometry, Journal of Agricultural and Food Chemistry, 51 (2003) 4624-4627.
J. H. Sietsma, A. B. Din, V. Ziv, K. A. Sjollema, O. Yarden, The localization of chitin synthase in membranous vesicles (chitosomes) in Neurospora crassa, Microbiology, 142 (1996) 1591-1596.
S. Demirci, N. Sahiner, The use of conductive polymers embedded macro porous pei and ionic liquid form of pei cryogels for potential conductometric sensor application to CO2, Journal of Composites Science, 4 (2020) 27.
N. Sahiner, S. Demirci, Poly ionic liquid cryogel of polyethyleneimine: Synthesis, characterization, and testing in absorption studies, Journal of Applied Polymer Science, 133 (2016).
B. Ari, B. Yetiskin, O. Okay, N. Sahiner, Preparation of dextran cryogels for separation processes of binary dye and pesticide mixtures from aqueous solutions, Polymer Engineering & Science, 60 (2020) 1890-1901.
S. V. Gohil, A. Padmanabhan, H.-M. Kan, M. Khanal, L. S. Nair, Degradation-dependent protein release from enzyme sensitive injectable glycol chitosan hydrogel, Tissue Engineering Part A, 27 (2021) 867-880.
A. Dal Pozzo, L. Vanini, M. Fagnoni, M. Guerrini, A. De Benedittis, R. Muzzarelli, Preparation and characterization of poly(ethylene glycol)-crosslinked reacetylated chitosans, Carbohydrate Polymers, 42 (2000) 201-206.
M. Fiol, A. Weckmüller, S. Neugart, M. Schreiner, S. Rohn, A. Krumbein, L. W. Kroh, Thermal-induced changes of kale's antioxidant activity analyzed by HPLC-UV/Vis-online-TEAC detection, Food chemistry, 138 (2013) 857-865.

1. A photocrosslinkable tissue bonding composition comprising: a poly(ethylene glycol) functionalized with at least one photocrosslinkable group, a hydroxy-modified chitosan, and a photochemically active dye.

2. The composition of claim 1, wherein the poly(ethylene glycol) functionalized with at least one photocrosslinkable group comprises poly(ethylene glycol) methyl ether acrylate, poly(ethylene glycol) dimethacrylate, poly(ethylene glycol), poly(ethylene glycol) diglycidyl ether, poly(ethylene glycol) diacrylate, poly(ethylene glycol) dithiol, poly(ethylene glycol) methyl ether thiol, poly(ethylene glycol) methyl ether amine, poly(ethylene glycol) bis(amine), or any combination thereof;
wherein the hydroxy-modified chitosan comprises glycol chitosan, hydroxy propyl chitosan, N,O-carboxymethyl chitosan, or any combination thereof; and
wherein the photochemically active dye comprises a Rose Bengal dye, Riboflavin, Eosin-Y, Erythrosine dye, or any combination thereof.

3-5. (canceled)

6. The composition of claim 1, wherein the composition further comprises 2-N-morpholinoethyl methacrylate, pentaerythritol tetraacrylate, pentaerythritol triacrylate, trimethylolpropane triacrylate, or any combination thereof.

7. The composition of claim 1, wherein the poly(ethylene glycol) functionalized with at least one photocrosslinkable group and the hydroxy-modified chitosan are fibers, films, medical textiles, microparticles, or nanoparticles.

8. The composition of claim 1, wherein the poly(ethylene glycol) functionalized with at least one photocrosslinkable group is a water-soluble precursor containing an epoxide, a thiol, an amine group, or any combination thereof.

9. The composition of claim 1, wherein the composition is cross-linkable at from 500 to 565 nm.

10. The composition of claim 1, wherein the composition further comprises an antibiotic, an antifungal, an anti-inflammatory medication, or any combination thereof.

11-13. (canceled)

14. The composition of claim 1, wherein the composition has a ratio of the poly(ethylene glycol) functionalized with at least one photocrosslinkable group: hydroxy-modified chitosan of from 1:20 to 1:1.

15. The composition of claim 1, wherein the poly(ethylene glycol) functionalized with at least one photocrosslinkable group has a poly(ethylene glycol) methyl ether amine:poly(ethylene glycol) diacrylate ratio of from 0:100 to 100:0.

16. The composition of claim 1, wherein the poly(ethylene glycol) functionalized with at least one photocrosslinkable group has a molecular weight of from 0.5 to 40 kDa; and wherein the hydroxy-modified chitosan has a molecular weight ranging from 540 to 810 g/mol.

17. (canceled)

18. A photocrosslinked tissue bonding composition comprising: a photochemically active dye and a poly(ethylene glycol), wherein the poly(ethylene glycol) comprises at least one group that is photocrosslinked, resulting in a photocrosslinked poly(ethylene glycol), and a hydroxy-modified chitosan entrapped in the photocrosslinked poly(ethylene glycol), grafted to the photocrosslinked poly(ethylene glycol), or any combination thereof.

19. The composition of claim 18, wherein the photocrosslinked poly(ethylene glycol) is a photocrosslinked poly(ethylene glycol) methyl ether acrylate, poly(ethylene glycol) dimethacrylate, poly(ethylene glycol), poly(ethylene glycol) diacrylate, poly(ethylene glycol) dithiol, poly(ethylene glycol) methyl ether thiol, poly(ethylene glycol) methyl ether amine, poly(ethylene glycol) diglycidyl ether, poly(ethylene glycol) bis(amine), or any combination thereof;
wherein the hydroxy-modified chitosan comprises glycol chitosan, hydroxy propyl chitosan, N,O-carboxymethyl chitosan, or any combination thereof; and
wherein the photochemically active dye comprises Rose Bengal dye, Riboflavin, Eosin-Y, Erythrosine dye, or any combination thereof.

20-21. (canceled)

22. The composition of claim 18, wherein the composition is an interpenetrating network or graft.

23. The composition of claim 18, wherein the hydroxy-modified chitosan is cross-linked with pentaerythritol tetraacrylate, pentaerythritol triacrylate, trimethylolpropane triacrylate, or any combination thereof.

24. (canceled)

25. The composition of claim 18, wherein the composition is soluble in blood and/or biocompatible.

26-27. (canceled)

28. The composition of claim 18, wherein the composition has a swelling percent of from 100% to 1000%, a moisture capacity of from 100% to 1000%, and/or a porosity percent of from 5% to 90%.

29-35. (canceled)

36. A method for producing a photocrosslinkable tissue bonding composition, comprising preparing a poly(ethylene glycol) functionalized with at least one photocrosslinkable group and a hydroxy-modified chitosan.

37. (canceled)

38. The method of claim 36, wherein the method further comprises combining the hydroxy-modified chitosan with an antibiotic, an antifungal, an anti-inflammatory medication, or any combination thereof.

39-41. (canceled)

42. A method for treating a wound, comprising contacting the wound with the composition of claim 1, and irradiating the composition with a light.

43. (canceled)

44. The method of claim 42, wherein the light is green light; and wherein irradiation occurs at from 500 to 565 nm.

45. (canceled)
